(19) United States
(12) Patent Application Publication
(10) Pub. No.: US 2010/0298728 A1
(43) Pub. Date: Nov. 25, 2010

(54) Signal Processing Techniques For Determining Signal Quality Using A Wavelet Transform Ratio Surface

(75) Inventors: Paul Stanley Addison, Edinburgh (GB); James Nicholas Watson, Dunfermline (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Mervue (IE)

(21) Appl. No.: 12/469,498

(22) Filed: May 20, 2009

(51) Int. Cl. A61B 5/02

(52) U.S. Cl. 600/504

(57) ABSTRACT

According to embodiments, a wavelet transform ratio surface measure signal may be generated from two PPG signals. Values of the wavelet transform ratio surface measure signal at a given moment of time (i.e., instantaneous values) may be indicative of localized signal discrepancies within and/or between the PPG signals such as noise and signal artifacts. Spikes in the instantaneous values of the wavelet transform ratio surface measure signal may be located and used to determine a signal quality measure for the PPG signals. Characteristics of the spikes such as number, location, grouping, distribution, amplitude, and polarity may be used in the signal quality determination.

### SUMMARY

The present disclosure is related to signal processing systems and methods, and more particularly, to systems and methods for processing photoplethysmograph (PPG) signals to determine signal quality.

In an embodiment, a wavelet transform ratio surface measure signal may be generated from two PPG signals (e.g., a red PPG signal and an infrared PPG signal). Values of the wavelet transform ratio surface measure signal at a given moment of time (i.e., instantaneous values) may be indicative of localized signal discrepancies within and/or between the PPG signals such as noise and signal artifacts. Spikes in the instantaneous values of the ratio surface measure signal may be located and used to determine a signal quality measure for the PPG signals. Characteristics of the spikes such as number, location, grouping, distribution, amplitude, and polarity may be used in the signal quality determination.

In an embodiment, a wavelet transform ratio surface measure signal may be used to determine signal quality. PPG signals may be obtained from any suitable source, such as a pulse oximeter sensor. The PPG signals may be transformed using continuous wavelet transforms to produce wavelet transformed signals. A wavelet transform ratio surface scalogram may be generated based on a ratio of the wavelet tranformed signal. A wavelet transform ratio surface measure signal may be generated from the wavelet transform ratio surface scalogram. For example, a desired feature such as a pulse region within the wavelet transform ratio surface scalogram may be identified and projected onto a time-amplitude plane to generate the ratio surface measure signal. Instantaneous values of the wavelet transform ratio surface measure signal may be used as a sensitive local indicator of signal quality and as a marker of noise. Relatively high energy areas (i.e., spikes) in the instantaneous values of the wavelet transform ratio surface measure signal may be located and may be used to determine a signal quality measure of the PPG signals.

Characteristics of the spikes located within the instantaneous values of the wavelet transform ratio surface measure signal may be indicative of signal quality. The number of spikes may be indicative of an amount and duration of noise or artifacts within the PPG signals. The location of the spikes may be indicative of the location of noise or artifacts within the PPG signals. The distribution of the spikes may be indicative of the cause or source of the noise or artifacts within the PPG signals. The amplitudes and amplitude variations of the spikes may be indicative of an amount of the noise or artifacts within the PPG signals. The polarity of the spikes (i.e., the direction, up or down) may be indicative of which of the two PPG signals may be associated with the noise or artifacts indicated by particular spikes.

In an embodiment, spikes located within in the instantaneous values of the wavelet transform ratio surface measure signal that are located within a particular duration and that share one or more characteristics may be grouped together. The characteristics of the groups of spikes may be considered as a whole when determining signal quality measures of the PPG signals. For example, the characteristics of a group of spikes may be defined by the duration of the group, the number of spikes within the group, amplitude and polarity of the signals within the group, or any other group metrics or statistics.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with embodiments;

FIG. 4 is a block diagram of an illustrative continuous wavelet processing system in accordance with some embodiments;

FIG. 5 is an illustrative plot of a scalogram derived from red photoplethysmograph (PPG) signal and an illustrative plot of a scalogram derived from an infrared PPG signal in accordance with some embodiments;

FIG. 6 is an illustrative plot of a wavelet transform ratio surface scalogram derived from the signals shown in the scalograms of FIG. 5 in accordance with some embodiments;

FIG. 7 shows a series of illustrative plots depicting the generation of a wavelet transform ratio surface measure signal from two PPG signals in accordance with some embodiments;

FIG. 8 is an illustrative plot of a wavelet transform ratio surface scalogram derived from the signals shown in FIG. 7 in accordance with some embodiments;

FIG. 9 shows another series of illustrative plots depicting the generation of a wavelet transform ratio surface measure signal from two PPG signals in accordance with some embodiments;

FIG. 10 depicts an illustrative process for determining signal quality using a wavelet transformation ratio surface measure signal; and

FIG. 11 depicts an illustrative process for locating groups of spikes within a wavelet transform ratio surface measure signal in accordance with an embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ) exp (−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)a combination of concentration and path length from emitter to detector as a function of time.

The traditional approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A-log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{d log I ( {λ}_{R} )}{\frac{d t}{\frac{d log I ( {λ}_{IR} )}{d t}}} ≃ \frac{I ( {t}_{2} , {λ}_{R} ) - I ( {t}_{1} , {λ}_{R} )}{\frac{I ( {t}_{1} , {λ}_{R} )}{\frac{I ( {t}_{2} , {λ}_{IR} ) - I ( {t}_{1} , {λ}_{IR} )}{I ( {t}_{1} , {λ}_{IR} )}}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

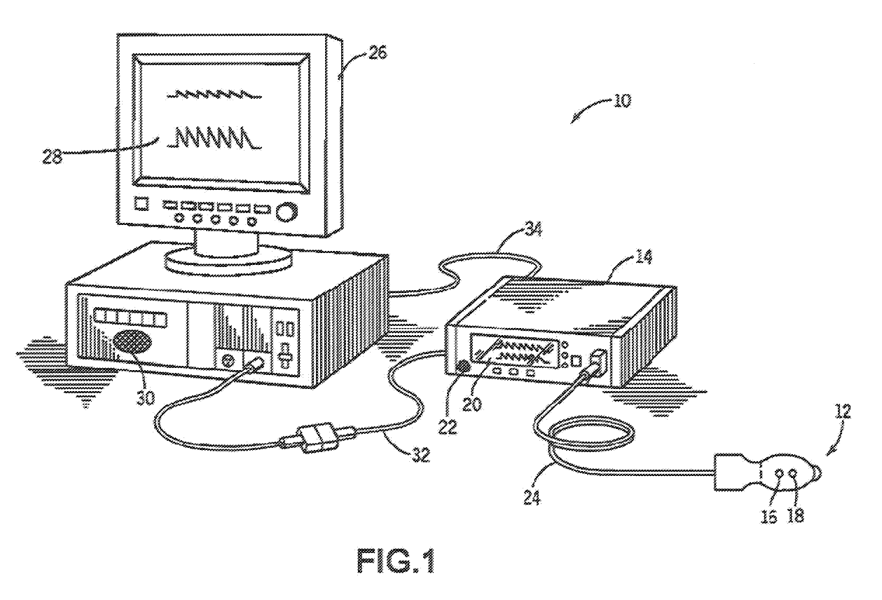

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

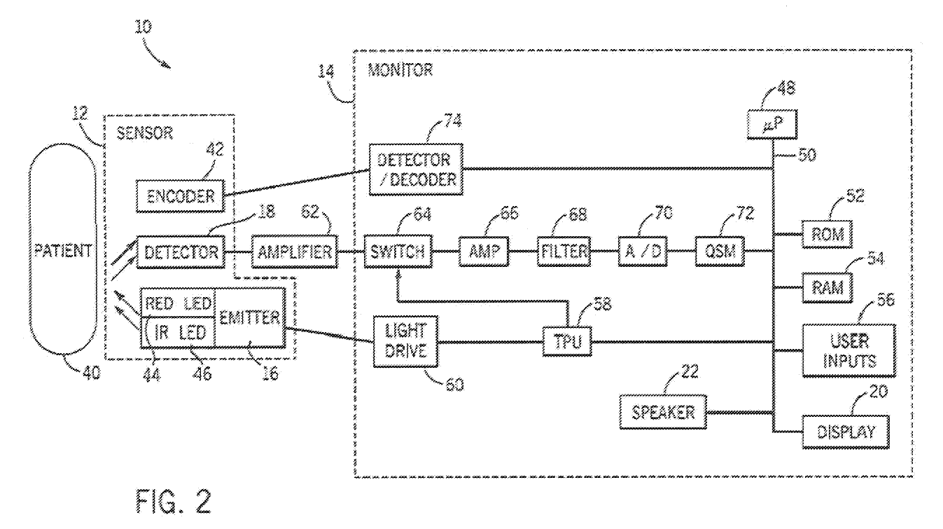

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patients tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely) operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be rescaled for useful purposes. One common resealing is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unscaled wavelet representation, linear rescaling, any power of the modulus of the wavelet transform, or any other suitable resealing. In addition, for purposes of clarity and conciseness, the term “scalogram” shall be taken to mean the wavelet transform, T(a,b) itself or any part thereof, For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{1/4}(e^{t2πf}^{₀}ₜ−e^{-(2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

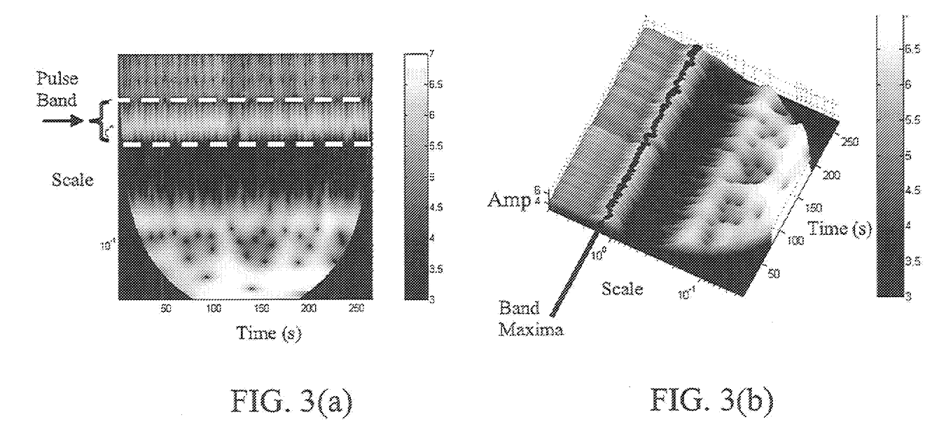

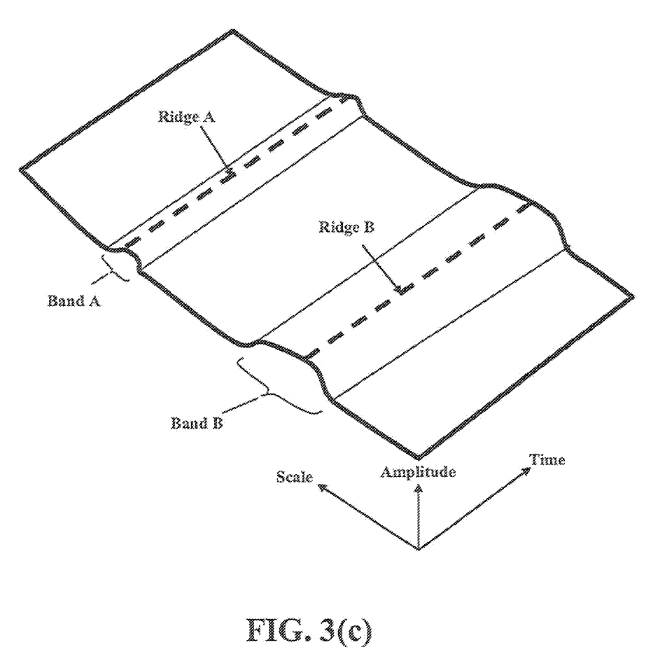

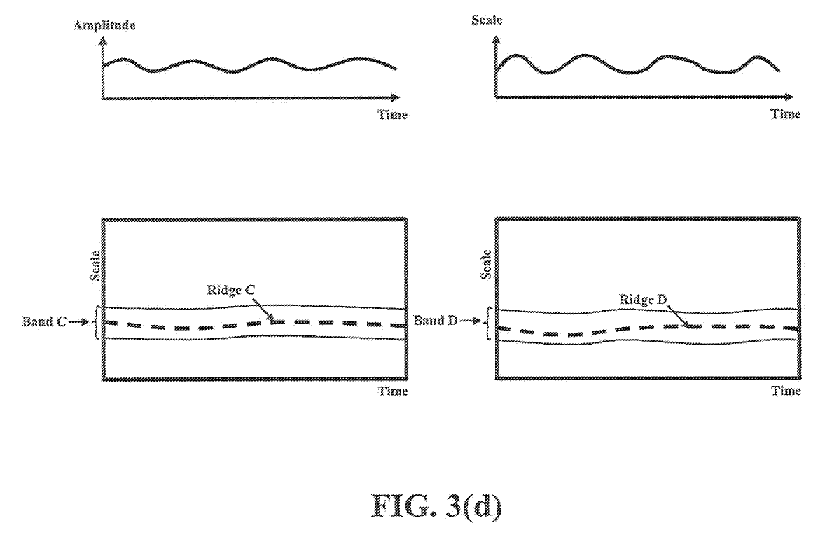

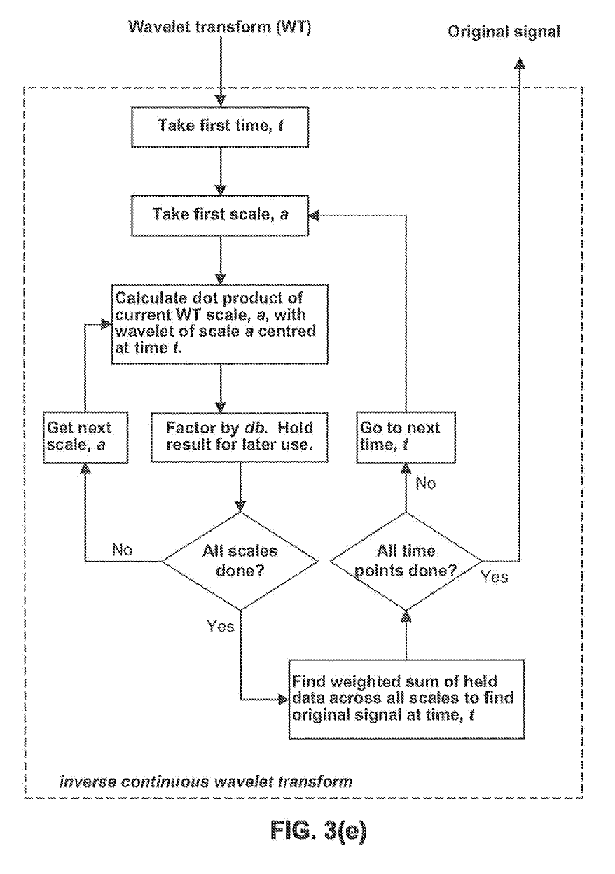

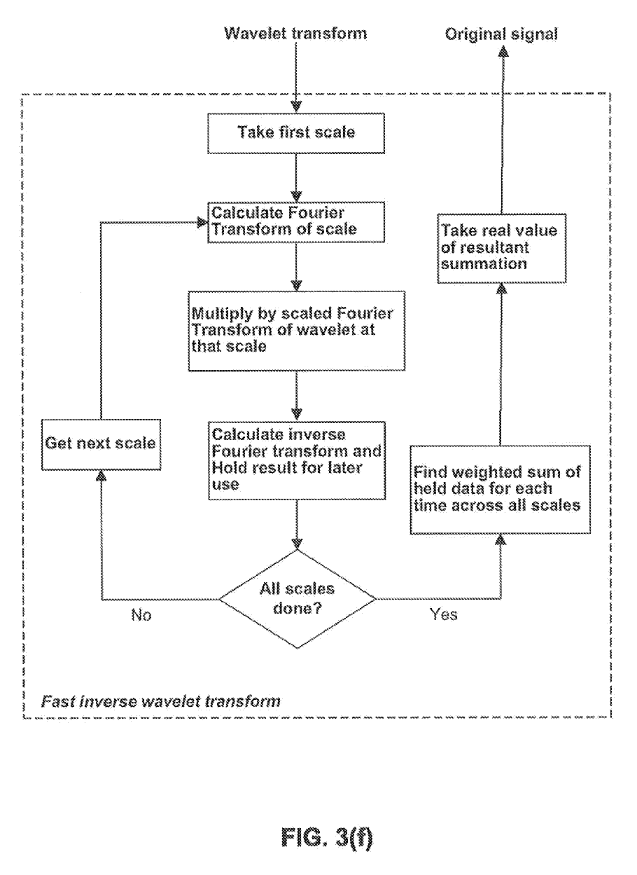

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

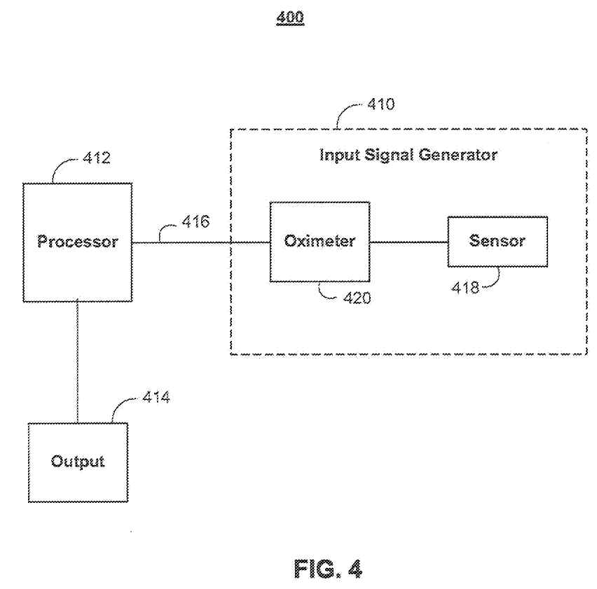

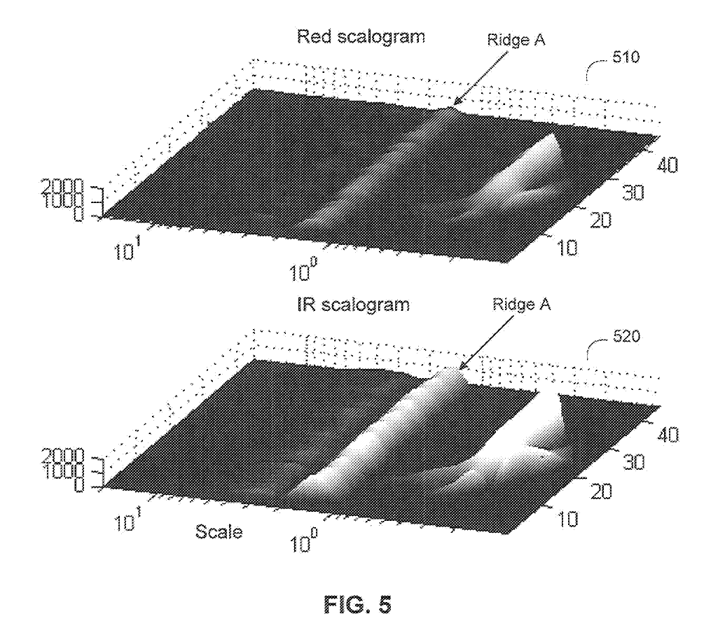

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

One technique for calculating blood oxygen saturation involves taking a continuous wavelet transform of a red PPG signal and of an infrared PPG signal and then computing a wavelet ratio surface. This technique is described in Addison et al., U.S. Patent Application Publication No. 2006/0258921, published on Nov. 16, 2006, which is hereby incorporated by reference herein in its entirety. This publication also describes another continuous wavelet transform-based technique for calculating blood oxygen saturation that involves generating a Lissajous figure in which the transformed (i.e., using a continuous wavelet transform) red and infrared signals are plotted with respect to one another.

FIG. 5 shows plots of two illustrative scalograms 510 and 520 derived from RED and IR PPG signals, respectively. For example, scalograms 510 and 520 may be derived using the same method (e.g., using continuous wavelet transforms) that was used to derive the scalograms shown in FIGS. 3(a), 3(b), and 3(c). The scalogram of the wavelet transform may be generated or otherwise obtained using, for example a processor such as processor 412 (FIG. 4) or microprocessor 48 (FIG. 2). The RED and IR PPG signals may be obtained using a sensor such as sensor 12 (FIG. 1). The sensor may include an emitter such as emitter 16 (FIG. 1, FIG. 2) and a detector such as detector 18 (FIG. 1, FIG. 2). The sensor emitter may include RED and IR light emitting sources such as RED LED 44 (FIG. 2) and IR LED 46 (FIG. 2). Ridge A indicated in plots 510 and 520 represent the locations of a ridge along a pulse band caused by the pulse component of the RED and IR PPG signals. This pulse band may be similar to pulse band A shown in FIG. 3(b).

A wavelet transform ratio surface (R_{WT}) can be constructed by dividing the wavelet transform of the logarithm of the RED PPG signal by the wavelet transform of the logarithm of the IR PPG signal to get a time-scale distribution of the wavelet transform ratio surface, i.e.

$${R}_{WT} = \frac{\vert {T ( a , b )}_{R} \vert}{\vert {T ( a , b )}_{1 R} \vert} \qquad ( 18 )$$

where the subscripts R and IR identify the RED and IR signals respectively. Note also that a wavelet transform ratio surface (R_{WT}) can be constructed by dividing wavelet transform of any mathematical manipulation of the original RED PPG signal by any mathematical manipulation of the wavelet transform of the IR PPG signal, for example, by dividing the wavelet transform of a normalized version of the RED PPG signal by the wavelet transform a normalized version of the IR PPG signal. Note also that a wavelet transform ratio surface (R_{WT}) can be constructed by dividing the wavelet transform of the original RED PPG signal by the wavelet transform of the original JR PPG signal.

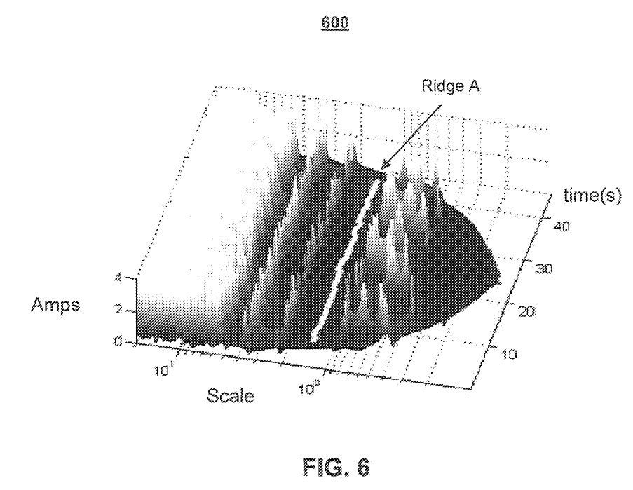

The wavelet transform ratio surface scalogram derived from the two scalograms in FIG. 5 is shown schematically in FIG. 6. Note that as described previously, a scalogram may include all reasonable forms of resealing including the original unscaled wavelet representation, linear resealing and any power of the modulus of the wavelet transform may be used in the definition. As the amplitude of the wavelet components scale with the amplitude of the signal components then for regions of the surface not affected by erroneous signal components the wavelet transform ratio surface will contain values which can be used to determine the oxygen saturation using a pre-defined look-up table. In order to obtain these values from regions of the surface that are not affected by erroneous signal components a characteristic value of the surface ratio may be used to determine the oxygen saturation. The characteristic value may be, for example, a mean value, weighted mean values, values at a certain percentile of a distribution of values, etc.

As can be seen in FIG. 6, the time-scale wavelet transform ratio surface along, and in close proximity to, the projection of the pulse ridge A onto the wavelet transform ratio surface is stable and may be used in the robust determination of the oxygen saturation. In an embodiment, the characteristic values obtained along the ridge A onto R_{WT} are used to determine oxygen saturation via a pre-defined look-up table which correlates R_{WT} to oxygen saturation.

The RED and IR PPG signals may contain erroneous or otherwise undesirable artifacts due to, for example, patient movement, equipment failure, and/or various noise sources. For example, cable 24, cable 32, and/or cable 34 (all of FIG. 1) may malfunction or become loosened from the equipment to which it is connected. Further, sensor 12 (FIG. 1), or any constituent component of sensor 12 (FIG. 1) (for example, emitter 16 (FIG. 1) and/or detector 18 (FIG. 1)) may malfunction and/or become loosened. Additionally, noise sources may produce inconsistent features in a PPG signal or other biosignal. Possible sources of noise include thermal noise, shot noise, flicker noise, burst noise, and/or electrical noise caused by light pollution. These and other noise sources may be introduced, for example, through sensor 12 (FIG. 1), and/or cables 24, 32, and 34 (all of FIG. 1). These and/or other phenomena may be present in a system such as pulse oximetry system 10 (FIG. 1), and thus may introduce inconsistent features into the measured PPG signal and in turn may introduce inconsistent features into the wavelet transform ratio surface. Values of the wavelet transform ratio surface that reflect these inconsistent features may be indicative of signal quality and noise within the RED and IR PPG signals.

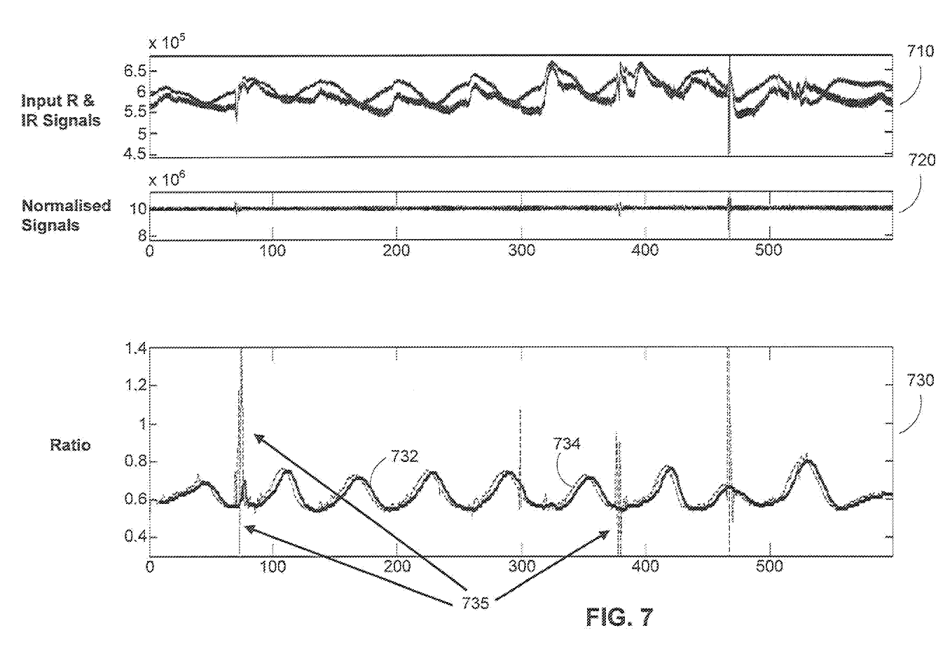

FIG. 7 shows a series of illustrative plots depicting the generation of a wavelet transform ratio surface measure signal in which time is represented on the x-axis and amplitude values are represented on the y-axis. Each of these plots may be displayed using any suitable display device such as, for example, monitor 20 (FIG. 1), display 28 (FIG. 1), a PDA, a mobile device, any other suitable display device, or multiple display devices. Plot 710 is an illustrative plot of both the RED and IR PPG signals. The PPG signals may be obtained from a patient, such as patient 40 (FIG. 2), using a sensor such as sensor 12 (FIG. 1). Alternatively, the PPG signals may be obtained by averaging or otherwise combining multiple signals derived from a suitable sensor array, as discussed in relation to FIG. 1. Further, PPG signals or any other related signal may be obtained from a source other than pulse oximeter system 10 (FIG. 1). For example, a PPG signal may be obtained from another type of medical device or from non-medical devices including a general signal oscilloscope and/or waveform analyzer. Plot 720 is an illustrative plot of normalized versions of the RED and IR PPG signals from plot 710. The PPG signals may be normalized by processing the signals in a pulse oximetry system such as pulse oximetry system 10 (FIG. 1) and may be carried out using a processor such as processor 412 (FIG. 4) or microprocessor 48 (FIG. 2).

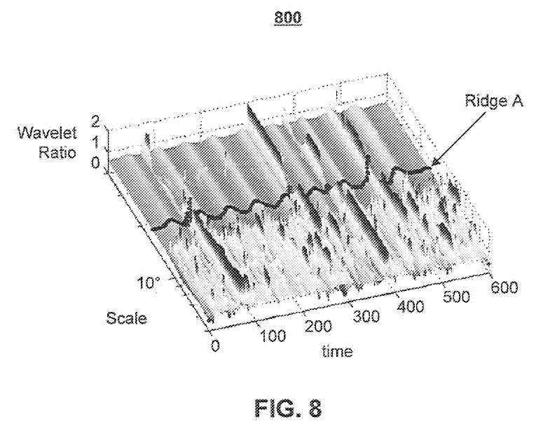

FIG. 8 shows an illustrative wavelet transform ratio surface scalogram plot 800 generated from the normalized PPG signals shown in plot 720. In an embodiment, a wavelet ratio surface plot may also be generated from the original PPG signals shown in plot 710. Wavelet transform ratio surface scalogram plot 800 may be generated using the same techniques described above with respect to FIGS. 5 and 6. Pulse ridge A within wavelet transform ratio surface scalogram plot 800 is indicated by the continuous black line.

Plot 730 of FIG. 7 is a plot of a wavelet transform ratio surface measure signal that may be generated by projecting pulse ridge A of wavelet ratio surface scalogram plot 800 onto a time-amplitude plane. Wavelet transform ratio surface signal 732 (solid line) represents the characteristic values of pulse ridge A of the wavelet transform ratio surface. Wavelet transform ratio surface measure signal 734 (dashed line) represents the instantaneous values of pulse ridge A of the continuous wavelet transform ratio surface, from which the characteristic values are derived. The instantaneous values of the pulse ridge may be the actual value of the ratio surface at a given moment of time. The wavelet transform ratio surface signal plots may be extracted from the wavelet transform ratio surface scalogram plot 800 (FIG. 8) using, for example, a processor such as processor 412 (FIG. 4) or microprocessor 48 (FIG. 2)

Spikes in the instantaneous values of wavelet transform ratio surface signal 734 such as spikes 735 may correspond to regions of artifacts or noise within the continuous wavelet transform ratio surface. The location of the spikes within signal 734 may be used to identify regions of noise within the normalized PPG signals shown in plot 720. The amplitude and polarity (i.e., its direction, up or down) of each spike within signal 734 may provide additional information on the nature of the local signal discrepancy that caused that spike.

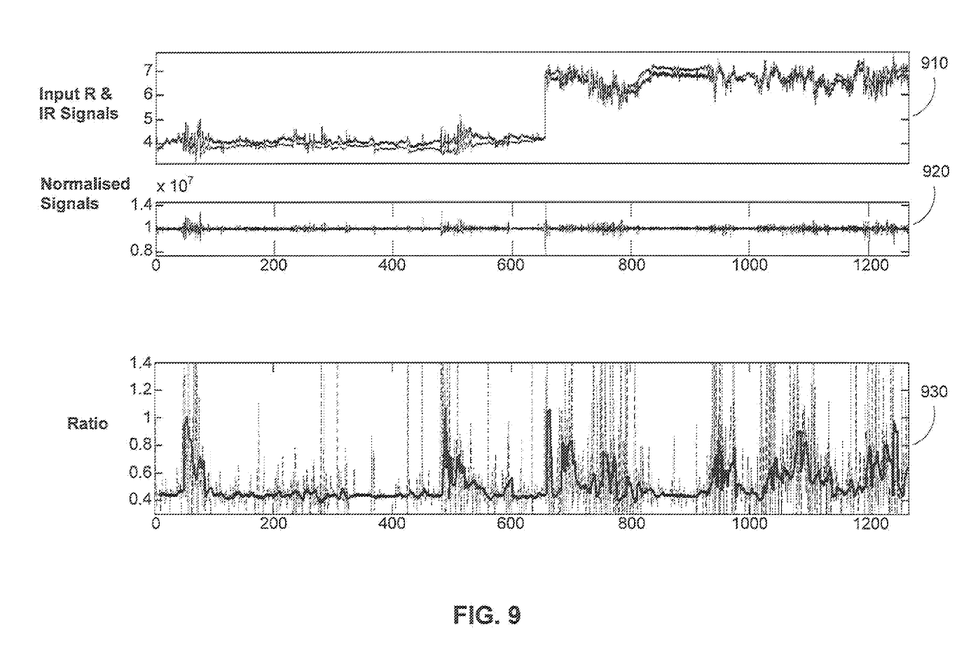

FIG. 9 shows another series of illustrative plots, similar to the plots in FIG. 7, in which the PPG signals (shown in plot 910) has an increased amount of noise (relative to plot 710 of FIG. 7). As a consequence of this increased amount of noise, it can be seen that plot 930 of a wavelet transform surface ratio measure signal derived from the normalized PPG signal shown in plot 920 has more spikes with greater amplitudes than counterpart plot 730 of FIG. 7.

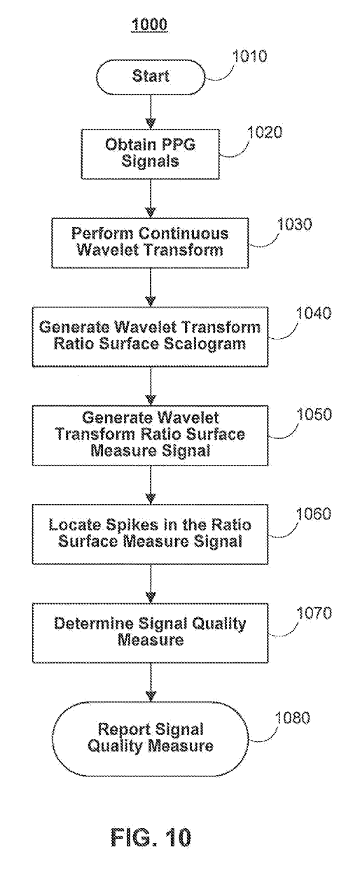

FIG. 10 depicts an illustrative process 1000 for determining the signal quality of received PPG signals using a wavelet transform ratio surface measure signal. Process 1000 may be implemented in a pulse oximetry system such as pulse oximetry system 10 (FIG. 1), and the steps of process 1000 may be carried out using a processor such as processor 412 (FIG. 4) or microprocessor 48 (FIG. 2).

Process 1000 may start at step 1010. At step 1020, process 1020 may obtain RED and IR PPG signals. The obtained signals may be PPG signals or any other suitable biosignal or general signal. The signal may be obtained from pulse oximetry system 10 (FIG. 1) using a sensor such as sensor 12 (FIG. 1) to measure biological characteristics of a patient such as patient 40 (FIG. 2). Additionally, the obtained signal may be a real-time signal or it may be a signal previously obtained and stored in memory, for example, ROM 52 (FIG. 2) or RAM 54 (FIG. 2).

The PPG signals obtained at step 1020 may be obtained by first receiving preliminary PPG signals and processing the preliminary PPG signals. The preliminary PPG signals may be obtained using, e.g., sensor 12 (FIG. 1) and processed using a processor such as processor 412 (FIG. 4) or microprocessor 48 (FIG. 2) in a system similar or identical to pulse oximetry system 10 (FIG. 1). The transformation of preliminary PPG signals into the signals obtained at step 1020 may include, for example, normalization, low-pass filtering, removal of noise-components, and/or interpolation methods to remove various undesirable artifacts that may be present in the preliminary signal.

At step 1030, the PPG signals obtained in step 1020 may be transformed (e.g., using a continuous wavelet transforms). Then, at step 1040 a wavelet transform ratio surface scalogram may be generated from the PPG signals transformed in step 1030. For example, the wavelet ratio surface scalogram may be derived using the same approach that is used to derive the scalograms shown in FIGS. 5 and 6 as well as the scalograms shown in FIGS. 3(a), 3(b), and 3(c). The wavelet transform ratio surface scalogram may be generated or otherwise obtained using, for example a processor such as processor 412 (FIG. 4) or microprocessor 48 (FIG. 2). In addition to the wavelet ratio surface scalogram, other parts of the wavelet transform may be determined. For example, the transform modulus, phase, real, and/or imaginary parts may be generated in addition to the scalogram. In an embodiment, scalograms of the RED and IR PPG signals may also be obtained. For example, scalograms of the RED and IR PPG signals may be processed to help generate the wavelet transform ratio surface scalogram and/or to help determine the features of the wavelet transform ratio surface scalogram. The generated scalograms may be displayed, for example, on monitor 26 (FIG. 1) or display 20 or 28 (both of FIG. 1).

The resultant wavelet transform ratio surface scalogram, generated in step 1040 may include bands and ridges corresponding to at least one area of increased energy. A pulse ridge may be identified from the wavelet transform ratio surface scalogram and/or from the PPG scalograms using, for example, a processor such as processor 412 (FIG. 4) or microprocessor 48 (FIG. 2), using any suitable method. The pulse ridge within these scalograms may be identified using characteristics of the scalogram including the energy and structure of the scalograms, and the signal-to-noise levels in various regions of scalograms. In one embodiment, this information may be calculated one or more times using different time-window sizes. The number and type of time-window sizes that are used may depend on the anticipated pulse rate, the available computational resources (e.g., the amount of ROM 52 (FIG. 2) and/or RAM 54 (FIG. 2) and the speed of processor 412 (FIG. 4) and/or microprocessor 48 (FIG. 2)), as well as on possible input derived from user inputs 56 (FIG. 2). For example, the pulse ridge identified from wavelet transform ratio surface scalogram may be similar to pulse ridges A within scalograms 600 and 800 of FIG. 6 and FIG. 8 respectively. Additionally or alternatively, the identification of pulse ridge A may be made or adjusted by a user or patient using, e.g., using user inputs 56 (FIG. 2). Pulse ridge A may be displayed, for example, on monitor 26 (FIG. 1) or display 20 or 28 (both of FIG. 1).

At step 1050 a wavelet transform surface measure signal may be generated from the identified pulse ridge in the wavelet transform ratio surface scalogram generated in step 1050. For example, the wavelet transform surface measure signal may be a plot of the identified pulse ridge within the wavelet transform ratio surface, projected onto the time-amplitude plane. The wavelet transform ratio surface measure signal may be generated from pulse ridges similar to pulse ridges A within scalograms 600 and 800 of FIG. 6 and FIG. 8 respectively. In an embodiment, the wavelet transform surface measure signal may be generated from transformed PPG signals produced at step 1030 and may not require the actual generation of a wavelet ratio surface scalogram. For example, a wavelet transform ratio surface measure signal may be generated from transformed normalized PPG signal of plots 730 (FIG. 7) and 830 (FIG. 8) Surface measure signal may be displayed, for example, on monitor 26 (FIG. 1) or display 20 or 28 (both of FIG. 1).

At step 1060 relatively high energy areas or spikes in the instantaneous values of the wavelet transform ratio surface measure signal may be located. Instantaneous values of the wavelet transform ratio surface measure signal are values of the wavelet transform ratio surface measure signal at a given moment in time. The instantaneous values of the wavelet transform ratio surface measure signal may differ from characteristic values of the wavelet transform ratio surface measure signal. Characteristic values of the wavelet transform ratio surface measure signal may be calculated based on a mean value, weighted mean value, median value, or a value at a certain percentile of a distribution of values of the ratio surface measure signal. While characteristic values of the ratio surface measure signal may be used to determine oxygen saturation levels, instantaneous values of the ratio surface measure signal may be used to measure signal quality of the PPG signals obtained in step 1020.

Spikes in the instantaneous values of the wavelet transform ratio surface measure signal may be a sharp rise followed by a sharp decline (or vise versa) in the wavelet transform ratio surface measure signal. Alternatively or additionally, the located spikes may include any set of instantaneous values of the wavelet transform ratio surface measure signal that differs from corresponding characteristic values of the ratio surface measure signal by more than a predetermined or calculated amount. In an embodiments, all of the spikes in the wavelet transform ratio surface measure signal or portion of the signal may be located. Alternatively, only spikes having amplitude values that exceed a particular amplitude threshold value may be located. In an embodiment, spikes may only be located at particular portions of the ratio surface measure signal.

The located spikes may represent erroneous or otherwise undesirable artifacts in the RED and IR PPG signals due to, for example, patient movement, equipment failure, and/or various noise sources. For example, cable 24, cable 32, and/or cable 34 (all of FIG. 1) may malfunction or become loosened from the equipment to which it is connected. Further, sensor 12 (FIG. 1), or any constituent component of sensor 12 (FIG. 1) (for example, emitter 16 (FIG. 1) and/or detector 18 (FIG. 1)) may malfunction and/or become loosened. Additionally, noise sources may produce inconsistent features in the PPG signals. Possible sources of noise include thermal noise, shot noise, flicker noise, burst noise, and/or electrical noise caused by light pollution. These and other noise sources may be introduced, for example, through sensor 12 (FIG. 1), and/or cables 24, 32, and 34 (all of FIG. 1). These and/or other phenomena may be present in a system such as pulse oximetry system 10 (FIG. 1), and thus may introduce inconsistent features into the measured PPG signals and in turn may introduce inconsistent features into the wavelet ratio surface.

Spikes may be located in the wavelet transform ratio surface measure signal, e.g., using any suitable signal processing technique, including a zero-crossing technique, a root-finding technique, an analytic curve-fitting technique, and/or a numerical analysis of the derivatives of the selected portion of the signal. These and other techniques may be implemented in pulse oximetry system 10 (FIG. 1) by processor 412 (FIG. 4), microprocessor 48 (FIG. 2), ROM 52 (FIG. 2), and/or RAM 54 (FIG. 2). Additionally, the parameters that may be used by suitable signal processing techniques, e.g., tolerance values and sensitivity levels, may be controlled by a user or patient using, e.g., using user inputs 56 (FIG. 2). Spikes that are located may be displayed, for example, on monitor 26 (FIG. 1) or display 20 or 28 (both of FIG. 1). Alternatively, a portion of the wavelet transform ratio surface measure signal may be displayed on a monitor, and a user may choose or otherwise influence which spikes are located using, for example, user inputs 56 (FIG. 2).

At step 1070 one or more signal quality measures of the PPG signals obtain in step 1020 may be determined based on the spikes in the instantaneous values of the ratio surface measure signal located in step 1070. The signal quality measures may be based on the number, location, grouping, distribution, amplitude, and polarity of the located spikes. The signal quality measure may be any suitable value. For example, the signal quality measure may be represented by a number from 1 to 100, where a larger number indicates a large amount of noise of artifacts in the signal (any other suitable number range could be used instead). The number may be indicative of the number, location, grouping, distribution, amplitude, and polarity of the located spikes in the signal. Additional signal quality measure may be provided to indicate individual signal criteria, for example, percentage of the signal affected. A signal quality measure may also indicate one or more potential sources of the noise or artifacts.

In an embodiment, the signal quality measure may be used to determined whether to include a corresponding portion of the PPG signals in an SpO₂ calculation. The signal quality measure may be calculated as the variance of the instantaneous values of the ratio surface—or a region of ratio surface. Other measurements may be specific percentile values of the instantaneous values of the ratio surface. The signal quality measure may also be used as the weight in the calculation of weighted means of physiological parameters, for example, SpO₂, pulse rate, fiducial point positions within a pulse, pulse peak amplitudes etc. The signal quality measures may also be used for the de-noising and or filtering of the PPG signals, for example, for weighted ensemble averaging of the PPG signals.

In an embodiment, the amplitude of each spike may correlate to an amount of noise or artifacts in the PPG signals. In an embodiment, spike amplitude threshold values may be used to analyze the located spikes. For example, only spikes that exceed a predetermined amplitude threshold value may be analyzed to determine signal quality. In another example, amplitude threshold values may be used to rank an amount of noise associated with each spike (e.g., a small, medium, or large amount of noise). In an embodiment, variations in amplitude value of the located spikes may be used to determine signal quality measures.

The signal quality measures may also be based on the polarities (i.e., the direction, up or down) of the identified spikes. The spikes represent an instantaneous value of the wavelet transform ratio surface measure signal at a particular location and because the wavelet transform ratio surface measure signal may be generated based on the ratio of the RED and IR PPG signals, the polarity of each spike may indicate which of the two PPG signals may be solely or primarily a source of the noise or artifact associated with the spike.

The signal quality measures may also be based on the locations of the spikes. For example, the location of a spike within the wavelet transform ratio surface measure signal may indicate the timing of noise or artifacts within the PPG signals. Additionally, the proximity in time of a spike with one or more other spikes within the ratio surface measure signal may also be calculated, for example, to determine the number of spikes located within a particular time period. Isolated spikes may be given less weight than, for example, a large cluster or group of spikes. Further, intermittency measures of the timing of the spikes may be used to detect patterns of noise or artifacts that may be used to determine possible sources of the noise or artifacts. For example, constant or persistent spikes may be indicative of a faulty sensor while a periodic spikes may be indicative of electrical noise. In this manner, the number, location, grouping, distribution, amplitude, polarity, or any other suitable characteristic of the spikes may be used to determine signal quality measures of the RED and IR PPG signals and in some instances to identify the source or sources of the noise or artifacts.

Finally, at step 1080 the signal quality measures determined in step 1070 may be reported. For example, the signal quality measures may be reported by generating an audible alert or, for example, using speaker 22 (FIG. 2) as well as possibly through other audio devices, generating an on-screen message, for example, on display 20 (FIG. 1) or display 28 (FIG. 1), generating a pager message, a text message, or a telephone call, for example, using a wireless connection embedded or attached to a system such as system 10 (FIG. 1), activating a secondary or backup sensor or sensor array, for example, connected through a wire or wirelessly to monitor 14 (FIG. 1), or regulating the automatic administration medicine, for example, which is controlled in part or fully through a system such as system 10 (FIG. 1). Additionally, the signal quality measures may be reported on a display such as display 20 (FIG. 1) or display 28 (FIG. 1) in graphical form using, for example, a bar graph or histogram. The signal quality measures may also be reported to one or more other processes, for example, to be used as part of or to improve the reliability of other measurements or calculations within a system such as pulse oximetry system 10 (FIG. 1).

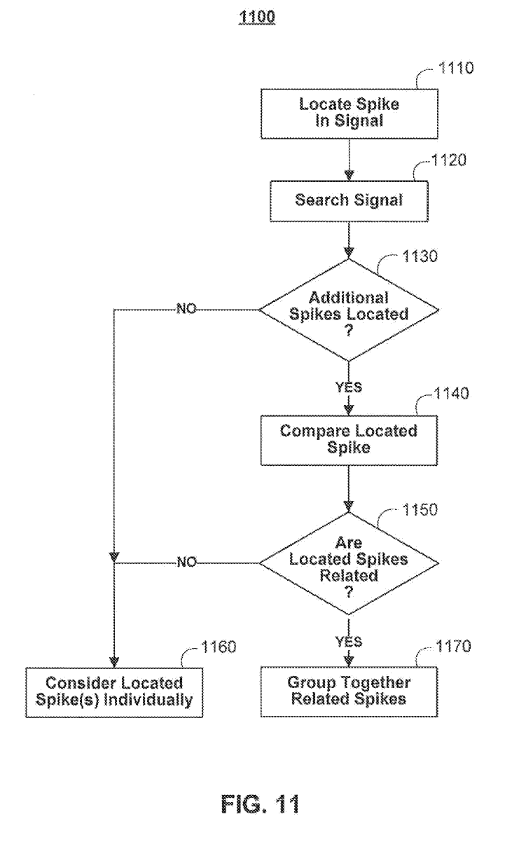

FIG. 11 depicts an illustrative process 1100 for identifying one or more groups of spikes within ratio surface measure signal. Process 1100 may be implemented in a pulse oximetry system such as pulse oximetry system 10 (FIG. 1), and the steps of process 1100 may be carried out using a processor such as processor 412 (FIG. 4) or microprocessor 48 (FIG. 2). Process 1100 may correspond to a further embodiment of process 1000 (FIG. 10), and more particularly, may correspond to a further embodiment of step 1060. In addition to locating individual spikes within a ratio surface measure signal in the manner described for step 1060 of FIG. 10, groups of spikes may be identifies. The spikes within a particular group may be analyzed as a single unit when determining the effect of the group of spikes on signal quality, for example at step 1070 of FIG. 10. The properties of a group of spikes that may be used to determine signal quality includes, for example, the location of the group, whether or not the group is repeated according to a pattern, the duration of the group, the amplitudes of the spikes within the group, the polarity of the spikes within the group, the number of the spikes within the group, and any other suitable group metric or statistic.

Process 1100 may start at step 1110. At step 1110, a signal peak within the ratio surface measure signal may be located. At step 1120 the wavelet transform ratio surface measure signal may be searched, before and after the signal peak located in step 1110. At step 1130 it may be determined whether or not additional spikes are located in step 1120. If no additional spikes are located, the spike located in step 1110 may be considered individually at step 1160. If additional spikes are found in step 1130, the properties of the located spikes may be compared at step 1140. At step 1150 it may be determined whether the located spikes are related (e.g., sharing one or more properties such as approximate amplitude values or polarity and/or occurring within a particular period of time). If the located spikes are not related, the spikes located at steps 1110 and 1120 may be considered individually at step 1160. If at least some of the spikes are related, at step 1170 the related spikes may be grouped together. The individual and groups of spikes determined using process 1110 may be used to determine signal quality, for example at step 1070 of FIG. 10

It will also be understood that the above method may be implemented using any human-readable or machine-readable instructions on any suitable system or apparatus, such as those described herein.

What is claimed is:

1. A method for determining signal quality using a wavelet transformation ratio surface measure, the method comprising:
obtaining a first photoplethysmograph (PPG) signal and a second PPG signal from a sensor;
performing a continuous wavelet transform of the first PPG signal and the second PPG signal to produce a first wavelet transformed signal and a second wavelet transformed signal;
generating a wavelet transform ratio surface scalogram based at least in part on a ratio of the first wavelet transformed signal to the second wavelet transformed signal;
generating a wavelet transform ratio surface measure signal from a region in the wavelet transform ratio surface scalogram; and
locating one or more relatively high energy areas in the wavelet transform ratio surface measure signal;
determining a signal quality measure for the first PPG signal and the second PPG signal based at least in part on the located relatively high energy areas in the wavelet transform ratio surface measure signal.

2. The method of claim 1 wherein the first PPG signal comprises a red PPG signal and the second PPG signal comprises an infrared PPG signal.

3. The method of claim 1, further comprising normalizing the first PPG signal and the second PPG signal obtained from the sensor prior to performing the continuous wavelet transforms.

4. The method of claim 1, wherein generating the wavelet transform ratio surface measure signal comprises identifying a pulse band within the wavelet transform ratio surface scalogram.

5. The method of claim 1, wherein the one or more relatively high energy areas located in the wavelet transform ratio surface measure signal are generally associated with noise in one of the first PPG signal and the second PPG signal at temporal locations corresponding to locations of the relatively high energy areas.

6. The method of claim 1, further comprising determining the signal quality measure based at least in part on a number of relatively high energy areas located in the wavelet transform ratio surface measure signal.

7. The method of claim 1, further comprising determining the signal quality measure based at least in part on an amplitude of the one or more relatively high energy areas located in the wavelet transform ratio surface measure signal.

8. The method of claim 1, further comprising determining the signal quality measure based at least in part on a polarity of the one or more relatively high energy areas located in the wavelet transform ratio surface measure signal.

9. The method of claim 8, wherein the polarity is based at least in part on a relative difference in a noise level associated with the first PPG signal and the second PPG signal.

10. The method of claim 1, further comprising determining that a plurality of the identified relatively high energy areas are part of a group of relatively high energy areas based at least in part on a proximity of the plurality of relatively high energy areas.

11. The method of claim 10, further comprising determining the signal quality measure based at least in part on an attribute of the group of relatively high energy areas.

12. The method of claim 1, further comprising determining a noise source based at least in part on the located relatively high energy areas.

13. A system for processing a signal to determine signal quality using a wavelet transformation ratio surface measure, the system comprising:
a sensor to receiving data indicative of a signal;
a processor coupled to the sensor, wherein the processor is capable of:
performing a continuous wavelet transform of the first PPG signal and the second PPG signal to produce a first wavelet transformed signal and a second wavelet transformed signal;
generating a wavelet transform ratio surface scalogram based at least in part on a ratio of the first wavelet transformed signal to the second wavelet transformed signal;
generating a wavelet transform ratio surface measure signal from a region in the wavelet transform ratio surface scalogram; and
locating one or more spikes in the wavelet transform ratio surface measure signal; and
determining a signal quality measure for the first PPG signal and the second PPG signal based at least in part on the located spikes in the wavelet transform ratio surface measure signal.

14. The system of claim 13, wherein the processor is further capable of normalizing the first PPG signal and the second PPG signal obtained from the sensor prior to performing the continuous wavelet transforms.

15. The system of claim 13, wherein the processor is further capable of identifying a pulse band within the wavelet transform ratio surface scalogram.

16. The system of claim 13, wherein the one or more spikes located in the wavelet transform ratio surface measure signal are generally associated with noise in one of the first PPG signal and the second PPG signal at temporal locations generally corresponding to locations of the spikes.

17. The system of claim 13, wherein the processor is further capable of determining the signal quality measure based at least in part on a number of spikes located in the wavelet transform ratio surface measure signal.

18. The system of claim 13, wherein the processor is further capable of determining the signal quality measure based at least in part on an amplitude of the one or more spikes located in the wavelet transform ratio surface measure signal.

19. The system of claim 13, wherein the processor is further capable of determining the signal quality measure based at least in part on a polarity of the one or more spikes located in the wavelet transform ratio surface measure signal.

20. The system of claim 19, wherein the polarity is based at least in part on a relative difference in a noise level associated with the first PPG signal and the second PPG signal.

21. The system of claim 13, wherein the processor is further configured to determine that a plurality of the identified spikes are part of a group of spikes based at least in part on a proximity of the plurality of spikes.

22. The system of claim 21, wherein the processor is further configured to determine the signal quality measure based at least in part on an attribute of the group of spikes.
